(19) United States
(12) Patent Application Publication
(10) Pub. No.: US 2025/0345740 A1
(43) Pub. Date: Nov. 13, 2025

(54) DEODORIZING FILTER FOR EXTERNAL ATTACHMENT OF FOOD WASTE DISPOSER TO PREVENT CONDENSATION

(71) Applicant: Hanmi Flexible Co., Ltd., Gimhae-si (KR)

(72) Inventor: Heon Pyeong JI, Busan (KR)

(73) Assignee: Hanmi Flexible Co., Ltd., Gimhae-si (KR)

(21) Appl. No.: 18/949,950

(22) Filed: Nov. 15, 2024

(30) Foreign Application Priority Data
May 10, 2024 (KR) 10-2024-0061729

(51) Int. Cl. B01D 53/04; A61L 9/014; A61L 9/16; A61L 101/02; B09B 3/60; B09B 101/70

(52) U.S. Cl. CPC B01D 53/0446; A61L 9/014; A61L 9/16; A61L 2101/02; A61L 2209/13; A61L 2209/14; B01D 2253/102; B01D 2257/90; B01D 2259/45; B09B 3/60; B09B 2101/70

(57) ABSTRACT

The present disclosure relates to a deodorizing filter for external attachment of a food waste disposer, including a housing, a storage tank, and a fan motor, wherein the deodorizing filter includes a filter front part including a front body, an air intake part, and an exhaust part, a filter rear part including a rear body, a divider rib part dividing the internal space between the front body and the rear body into an upper storage space and a lower exhaust space, and activated carbon stored in the upper storage space. Accordingly, by applying expanded polypropylene to the filter rear part exposed to the outside, and having the divider rib part to provide the lower exhaust space in order to facilitate air circulation of the fan motor, it is 10 possible to prevent condensation from occurring even in temperature changes between a food waste disposer and the outside.

### CROSS-REFERENCE TO RELATED PATENT APPLICATION

The present application claims priority under 35 U.S.C. § 119(a) to Korean patent application number 10-2024-0061729 filed on May 10, 2024, in the Korean Intellectual Property Office, the entire disclosure of which is incorporated by reference herein.

### BACKGROUND OF THE INVENTION

### 1. Field

Embodiments of the present disclosure relate to a deodorizing filter for external attachment of a food waste disposer to prevent condensation, and more specifically, to a deodorizing filter for external attachment of a food waste disposer, to which expanded polypropylene, an eco-friendly insulation material, is applied and a lower exhaust space to facilitate air circulation of a fan motor is provided, thereby preventing condensation.

### 2. Description of the Related Art

In general, food waste produced after eating and cooking at home or restaurants is managed separately from general waste due to environmental pollution and hygiene issues. In other words, hygienic management and disposal are required by making food waste collected separately from general waste, or by differentiating costs depending on the amount of food waste discharged. In addition, in the case of apartment buildings, since the collection place for food waste is generally designated in a specific outdoor place, due to the hassle from frequent carrying over, there are many cases that the food waste in the household is often stored until a certain amount is reached and then disposed at once, causing a negative impact on the indoor environment.

Due to these management and economic reasons, methods of self-disposal of food waste indoors have recently been developed, one of which in frequent use is an eco-friendly food waste disposer that temporarily stores food waste in storage rooms where a condition for microorganism activation is created, followed by disposal of the food waste after it is naturally decomposed by microorganisms. While such food waste disposers mainly use hot air at high or low temperatures to dry and reduce food, there has been a problem that the odor generated in this process is discharged to the outside of the food waste disposer.

To solve this problem, the recently developed food waste disposer includes a deodorizing filter formed of activated carbon, which has a function of adsorbing and removing the odor by activated carbon by discharging the odor generated inside the food waste disposer after passing through the activated carbon charged on the inside. As such, the sales of the food waste disposers including activated carbon have been on a sharp rise recently as they are movable in use without restrictions to be installed near sewers.

However, the food waste disposers that are currently on the market are placed close to the outside so that the filter that supports the activated carbon directly receives the external temperature, resulting in condensation occurring on the activated carbon due to the temperature difference between a storage tank and the outside during winter. Moreover, as the fan motor for discharging the air inside the food waste disposer to the outside is placed near the activated carbon, the flow of air is interrupted to cause moisture to condense easily in the activated carbon near the fan motor, leading to condensation as well.

### PRIOR-ART DOCUMENT

### Patent Document

Korean Patent No. 10-0817318

### SUMMARY OF THE INVENTION

To solve the above problem, embodiments of the present disclosure provide a deodorizing filter for external attachment of a food waste disposer, to which expanded polypropylene, an eco-friendly insulation material, is applied and a lower exhaust space is provided to facilitate air circulation of a fan motor, thereby preventing condensation.

The object of the present disclosure is achieved by a deodorizing filter for external attachment of a food waste disposer to prevent condensation, wherein the deodorizing filter for external attachment of a food waste disposer, including a housing, a storage tank installed inside the housing to store and dispose food waste, and a fan motor installed at a lower side of the storage tank to discharge air in the storage tank to the outside, includes: a filter front part including a front body arranged toward the storage tank and installed on an outer surface of the housing, an air intake part formed in an upper part of the front body to receive the air discharged from the storage tank, and an exhaust part formed in a lower part of the front body to allow air sucked in through the air intake part to be discharged to the fan motor; a filter rear part including a rear body that is installed to face the front body to form an internal space; a divider rib part installed in a horizontal direction between the front body and the rear body to divide the internal space into an upper storage space and a lower exhaust space; and activated carbon stored in the upper storage space.

Herein, it is preferable that the filter rear part is formed of an expanded polypropylene (EPP) material, and it is preferable that the filter rear part has a thickness of 8 to 12 mm, and the expanded polypropylene is foamed in a foaming ratio of 8 to 10 times.

In addition, it is preferable that the divider rib part has a plurality of through holes formed therein, or the divider rib part includes a rib body supported by the front body or the rear body and a plurality of protrusions protruding from the rib body, or it is preferable that the divider rib part is detachable from the front body or the rear body.

As described above, according to the present disclosure, by applying expanded polypropylene, an eco-friendly insulation material, to a filter rear part exposed to the outside, it is possible to prevent condensation from occurring even in temperature changes between a food waste disposer and the outside and achieve an effect of preventing condensation by having a divider rib part to provide a lower exhaust space in order to facilitate air circulation of a fan motor.

### BRIEF DESCRIPTION OF THE DRAWINGS

FIG. 1 shows a cross-sectional diagram (A) illustrating a deodorizing filter of a food waste disposer according to a related art, and a cross-sectional diagram (B) of a deodorizing filter for external attachment of a food waste disposer according to an embodiment of the present disclosure.

FIGS. 2 to 4 are exploded perspective views of a deodorizing filter for external attachment of a food waste disposer according to embodiments of the present disclosure.

### DETAILED DESCRIPTION

Hereinafter, the technical idea of the present disclosure is described in detail with reference to the accompanying drawings. The drawings are merely examples shown to describe the technical idea of the present disclosure more specifically, and the technical idea of the present disclosure is not limited to the forms of the accompanying drawings.

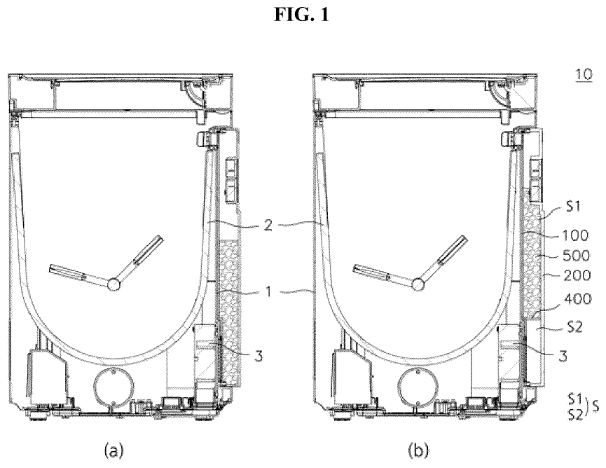

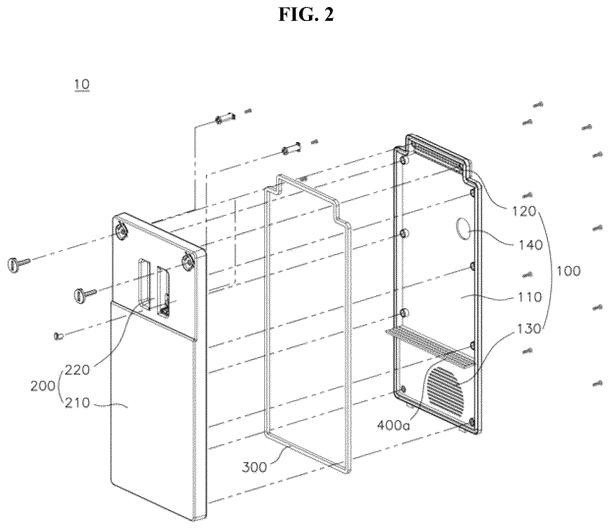

FIG. 1(A) is a cross-sectional diagram illustrating a deodorizing filter of a food waste disposer according to the related art, FIG. 1(B) is a cross-sectional diagram of a deodorizing filter for external attachment of a food waste disposer according to an embodiment of the present disclosure, and FIGS. 2 to 4 are exploded perspective views of a deodorizing filter for external attachment of a food waste disposer according to embodiments of the present disclosure.

The present disclosure relates to a deodorizing filter 10 that is attachable to the outside of a food waste disposer, and the deodorizing filter 10 for external attachment of a food waste disposer to prevent condensation according to an embodiment of the present disclosure is used in food waste disposers including, as shown in FIG. 1, a housing 1, a storage tank 2 installed inside the housing to store and dispose food waste, and a fan motor 3 installed at a lower side of the storage tank 2 to discharge the air in the storage tank 2 to the outside. In other words, while odor generated from the storage tank 2 where food waste is disposed is not discharged to the outside as it is, by installing the deodorizing filter 10 between the storage tank 2 and the fan motor 3, the odor generated from the storage tank 2 is purified through the deodorizing filter 10, and then the purified air is finally discharged to the outside through the fan motor 3.

The deodorizing filter 10 for external attachment of a food waste disposer to purify the odor generated from the food waste disposer includes a filter front part 100, a filter rear part 200, a gasket part 300, a divider rib part 400, and activated carbon 500 as shown in FIG. 1(B) and FIG. 2.

First, the filter front part 100 has a configuration to be arranged toward the storage tank 2 and connected to the storage tank 2, including a front body 110, an air intake part 120, an exhaust part 130, and an activated carbon insertion part 140.

The front body 110 has a configuration to be arranged toward the storage tank 2 and installed on an outer surface of the housing 1 and is formed in a plate shape and installed on the outer surface of the housing 1. Specifically, the housing 1 of the food waste disposer is formed in a shape with a rear surface recessed so that the deodorizing filter 10 is installed, and the front body 110 of the deodorizing filter 10 is installed on the recessed rear surface of the housing 1. In other words, the recessed rear surface of the housing 1 may be formed in a shape similar to the front body 110, the front body 110 may be installed to be fitted to the housing 1. It is preferable that such front body 110 be selected from the group consisting of polycarbonate (PC), acrylonitrile-butadiene-styrene (ABS), and a mixture thereof.

Polycarbonate has excellent mechanical properties such as strength and heat resistance, and ABS has the advantages such as excellent impact resistance and chemical resistance, all of which are easy to process using injection molding and compatible with other resins to be easily applied to the front body 110, but various resins are applicable without limitation.

The air intake part 120 has a configuration to be formed in an upper part of the front body 110 to receive air discharged from the storage tank 2, and includes a plurality of air intake holes. The air intake part 120 is connected to an upper part of the storage tank 2 where air is discharged from the storage tank 2, so that the discharged air passes through the air intake part 120 and enters an internal space(S) to be described later.

The exhaust part 130 has a configuration formed in a lower part of the front body 110 so that air sucked in through the air intake part 120 is discharged to the fan motor 3, and has a diameter similar to the diameter of the fan motor 3 and a shape such that the air discharged toward the fan motor 3 is discharged uniformly throughout the fan motor 3. In other words, the exhaust part 130 is formed in the front body 110 in an area facing the fan motor 3, is configured in a circular shape similar to the shape of the fan motor 3, and includes a plurality of exhaust holes each formed in a long shape in the horizontal direction. In this way, the air discharged through the exhaust part 130 is discharged to the outside of the food waste disposer through the fan motor 3.

The activated carbon insertion part 140 has a configuration to be formed in the front body 110 to insert the activated carbon 500 and is formed close to the upper part of the front body 110, but lower than the air intake part 120. The activated carbon insertion part 140 may allow the activated carbon 500 that deodorizes air to be inserted, and if the performance of activated carbon 500 decreases by repeated use, new activated carbon 500 may be inserted after removing the activated carbon 500.

The filter rear part 200 has a configuration to be exposed to the outside from the housing 1 and includes a rear body 210 and a handle part 220.

The rear body 210 has a configuration to form the internal space(S) by being installed to face the front body 110, and is formed in a structure in which the circumference of the rear body 210 protrudes in a ‘⊏’ shape toward the front body 110 so that the internal space(S) is formed in combination with the front body 110 formed in a plate shape. In other words, the rear body 210 is formed in a plate shape and has a structure in which its outer surface protrudes, and when combined with the front body 110 in the plate shape, the internal space(S) is formed between the front body 110 and the rear body 210.

The handle part 220 is formed in an upper part of the rear body 210 and formed on a surface of the rear body 210 facing outward such that a user may grasp the deodorizing filter 10 and attach or detach the deodorizing filter 10 from the food waste disposer. It is preferable that the handle part 220 is formed by being recessed toward the rear body 210 so that the user may put his or her hand in the recessed area and hold it, but it is not limited thereto, and any shape that allows the user to hold the handle part 220 may be applied without limitation.

When the food waste disposer is used in winter or at a low temperature, condensation occurs in the deodorizing filter 10 due to the temperature difference between the food waste disposer and the outside. When condensation occurs in the deodorizing filter 10 as described above, water accumulates in the deodorizing filter 10 and degrade the deodorization performance, and the water enters the food waste disposer by repeated condensation, causing malfunction of the food waste disposer. Therefore, in order to prevent such condensation, it is preferable that the filter rear part 200 is formed of expanded polypropylene (EPP), an eco-friendly insulation material.

The expanded polypropylene refers to one that is formed by foaming polypropylene (PP), which is not sensitive to external temperature changes, in a medium strength between plastic and Styrofoam, and by applying polypropylene, it is possible to make it insensitive to temperature changes between the food waste disposer and the outside, while preventing condensation from occurring by foaming the polypropylene to form an air gap in the filter rear part 200.

At this time, it is preferable that the filter rear part 200 formed of the expanded polypropylene be formed to have a thickness of 8 to 12 mm, which is thicker than the filter front part 100, and if the thickness of the filter rear part 200 is less than 8 mm, the filter rear part 200 may not be able to form the air gap sufficiently to prevent condensation, and if the thickness of the filter rear part 200 exceeds 12 mm, due to high thickness, aesthetic appearance degrades when installing the deodorizing filter 10 in the food waste disposer.

In addition, it is preferable that the filter rear part 200 is foamed with the expanded polypropylene in a foaming ratio of 8 to 10 times, which is significantly lower than the foaming ratio of 40 to 50 times that is the rate that Styrofoam is typically foamed, so as to derive the strength between Styrofoam and plastic. If the foaming ratio of expanded polypropylene is less than 8 times, it is difficult to prevent condensation because the air gap in the filter rear part 200 is not sufficiently formed, and if the foaming ratio exceeds 10 times, the strength of the filter rear part 200 is reduced to degrade the durability of the deodorizing filter 10.

The gasket part 300 is installed between the filter front part 100 and the filter rear part 200 to play a role in sealing the internal space(S), and it functions to seal so that moisture or air from the outside does not flow into or discharge from the internal space(S).

The divider rib part 400 is installed in a horizontal direction between the front body 110 and the rear body 210 and serves to divide the internal space(S) into an upper storage space (S1) and a lower exhaust space (S2), and also functions to divide the storage space(S) for the activated carbon so that the activated carbon 500 to be described later does not block the exhaust part 130 to allow the air to be discharged smoothly. Since the conventional deodorizing filter does not include a separate divider rib part as shown in FIG. 1(A), activated carbon is placed to the bottom of the internal space, thereby closing the front of the fan motor 3 when installing the deodorizing filter in the food waste disposer. As such, if the front of the fan motor 3 is blocked by activated carbon, the air in the activated carbon cannot escape to the outside, causing condensation, and in severe cases, generating a bad odor.

Therefore, in order to prevent this, in the present disclosure, the divider rib part 400 is used to divide the internal space(S) into the upper storage space (S1) and the lower exhaust space (S2), the activated carbon 500 is placed in the upper storage space (S1), and a space is provided to allow air to freely flow with no activated carbon 500 placed in the lower exhaust space (S2), thereby preventing condensation from occurring in the activated carbon 500. Here, to prevent the activated carbon 500 from blocking the exhaust part 130, it is preferable that the divider rib part 400 is installed at a height ratio of the upper storage space (S1): the lower exhaust space (S2)=2 to 3:1 for the total height of the front body 110 and rear body 210, and if the divider rib part 400 is installed at a higher height than this, it is difficult to store a large amount of activated carbon 500 to lead to reduction in the deodorization efficiency of the deodorizing filter 10, and if the divider rib part 400 is installed at a lower height, the activated carbon 500 may block the exhaust part 130.

The divider rib part 400a may be formed in a structure with a plurality of through holes formed as shown in FIG. 2, and here, it is preferable that the through hole be formed to have a diameter of 1 to 2 mm so that activated carbon 500 does not escape to the lower exhaust space (S2).

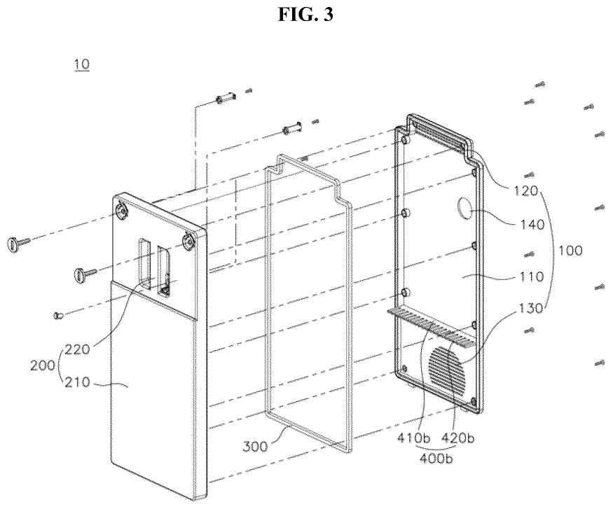

In addition, the divider rib part 400b may be formed in a structure including a rib body 410b supported on the front body 110 or rear body 210 and a plurality of protrusions 420b protruding from the rib body 410b, as shown in FIG. 3. Here, the plurality of protrusions 420b may have a width of 2 to 4 mm respectively, and the spacing between the protrusions 420b is configured to be 1 to 2 mm to prevent the activated carbon 500 from escaping to the lower exhaust space (S2).

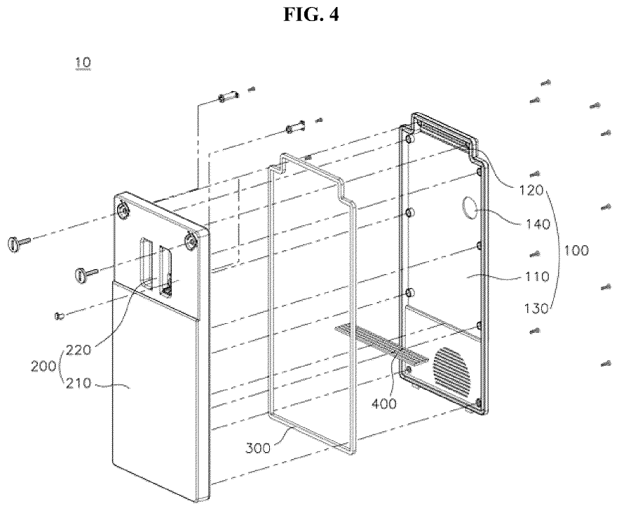

In some cases, the divider rib part 400 may be formed in a structure detachable from the front body 110 or the rear body 210 for ease of cleaning of the internal space(S) after separating the filter front part 100 and the filter rear part 200 as shown in FIG. 4.

The activated carbon 500 is stored in the upper storage space (S1) to play a role in deodorizing the air flowing into the internal space(S), may be entered into the upper storage space (S1) through the activated carbon insertion part 140 formed in the filter front part 100, and if replacement is required, may be removed through the activated carbon insertion part 140 to introduce new activated carbon 500. It is preferable that the activated carbon 500 have a diameter of 3 to 4 mm so as not to pass between the divider rib parts 400 while being able to deodorize air smoothly.

As such, if the deodorizing filter 10 for the external attachment of a food waste disposer to prevent condensation according to the present disclosure is installed in the food waste disposer, the deodorizing filter 10 may be removed from the food waste disposer if necessary to replace the activated carbon 500, thereby increasing the maintenance period of the food waste disposer. In addition, in order to prevent condensation that occurs when using the food waste disposer in winter or at low temperature, by applying expanded polypropylene, an eco-friendly insulation material, to the filter rear part 200 exposed to the outside, it is possible to prevent condensation from occurring even in the temperature change between the food waste disposer and the outside, and by having the divider rib part 400 to provide the lower exhaust space (S2) in order to facilitate the air circulation of the fan motor 3, it is possible to achieve an effect of preventing condensation.

The present disclosure is not limited to the above embodiments, the scope of application is diverse, and it is apparent that various modifications can be made without departing from the gist of the present disclosure claimed by the appended claims.

### EXPLANATION OF SYMBOLS

S: Internal space
S1: Upper storage space
S2: Lower exhaust space
1: Housing
2: Storage tank
3: Fan motor
10: Deodorizing filter
100: Filter front part
110: Front body
120: Air intake part
130: Exhaust part
140: Activated carbon insertion part
200: Filter rear part
210: Rear body
220: Handle part
300: Gasket part
400, 400a, 400b: Divider rib parts
410b: Rib body
420b: Protrusion
500: Activated carbon

What is claimed is:

1. A deodorizing filter for external attachment of a food waste disposer that comprises a housing, a storage tank installed inside the housing to store and dispose food waste, and a fan motor installed at a lower side of the storage tank to discharge air in the storage tank to the outside, the deodorizing filter comprising:
a filter front part comprising a front body arranged toward the storage tank and installed on an outer surface of the housing, an air intake part formed in an upper part of the front body to receive the air discharged from the storage tank, and an exhaust part formed in a lower part of the front body to allow air sucked in through the air intake part to be discharged to the fan motor;
a filter rear part comprising a rear body that is installed to face the front body to form an internal space;
a divider rib part installed in a horizontal direction between the front body and the rear body to divide the internal space into an upper storage space and a lower exhaust space; and
activated carbon stored in the upper storage space.

2. The deodorizing filter according to claim 1, wherein the filter rear part is formed of an expanded polypropylene (EPP) material.

3. The deodorizing filter according to claim 2, wherein the filter rear part has a thickness of 8 to 12 mm, and the expanded polypropylene is foamed in a foaming ratio of 8 to 10 times.

4. The deodorizing filter according to claim 1, wherein the divider rib part has a plurality of through holes formed therein.

5. The deodorizing filter according to claim 1, wherein the divider rib part comprises a rib body supported by the front body or the rear body, and a plurality of protrusions protruding from the rib body.

6. The deodorizing filter according to claim 1, wherein the divider rib part is detachable from the front body or the rear body.
